(19) United States
(12) Patent Application Publication
(10) Pub. No.: US 2025/0302366 A1
(43) Pub. Date: Oct. 2, 2025

(54) METHODS FOR DETERMINING THE RELAXING OR STIMULATING PROPERTIES OF A FRAGRANCE OR AROMA

(71) Applicant: FIRMENICH SA, Satigny (CH)

(72) Inventors: Sylvain DELPLANQUE, Genève (CH); David SANDER, Genève (CH); Christian MARGOT, Belmont-sur-Lausanne (CH); Christelle PORCHEROT LASSALLETTE, Genève (CH); Donato CEREGHETTI, Genève (CH)

(73) Assignee: FIRMENICH SA, Satigny (CH)

(21) Appl. No.: 18/723,689

(22) PCT Filed: Dec. 21, 2022
(86) PCT No.: PCT/EP2022/087259

§ 371 Date: Jun. 24, 2024

(30) Foreign Application Priority Data
Dec. 23, 2021 (EP) 21217360.3
May 31, 2022 (EP) 22176541.5

(51) Int. Cl. A61B 5/00; A61B 5/0205; A61B 5/024; A61B 5/0533; A61B 5/16; A61M 21/00; G16H 50/30

(52) U.S. Cl. CPC A61B 5/4035; A61B 5/0205; A61B 5/02405; A61B 5/02416; A61B 5/0533; A61B 5/165; A61B 5/7271; A61M 21/00; G16H 50/30; A61M 2021/0016

(57) ABSTRACT

The present invention relates to the field of fragrances and aromas. More specifically, the present invention relates to methods for determining the medium term effects of an odor or aroma using an index that reveals the relaxing/stimulating (energizing, invigorating) properties of the odor or aroma on the autonomic nervous system.

### FIELD OF THE INVENTION

The present invention relates to the field of fragrances and aromas. More specifically, the present invention relates to methods for determining the medium term effects of an odor or aroma using an index that reveals the relaxing/stimulating (energizing, invigorating) properties of the odor or aroma on the autonomic nervous system.

### BACKGROUND OF THE INVENTION

Emotions and affective states elicited by fragrances and aromas are important drivers of consumer satisfaction. Tools relying on oral consumer declarations have been used to characterize the conscious aspects of fragrance-elicited emotions.

Methods have been attempted to reveal unconscious aspects of fragrance-elicited emotions, especially through the observation of physiological parameters. Proposed measurements of fragrance-induced physiological parameters include changes of electrical brain activity through EEG, cardiac rhythm through ECG, dermal conductance etc. These event-related methods are often unsatisfactory for the evaluation of smell because of the poor temporal resolution offered by most olfactory stimulation techniques. Further, these works in olfaction utilize reverse-inference, i.e. Identify an emotional state based on physiological readings obtained in a very limited study. Existing published scientific studies report on investigations of the relationship between verbally reported relaxing/energizing properties of odors and physiological states when the subjects smell the odors under study. For example, Loos et al., (2020) describes event-related responses that represent the immediate physiological adaptation to the stimulation. However, the attempts so far cannot provide information on the medium term effects of smells and there is no reliable information on whether the relaxing or stimulating effects of certain smells last when the smell is no longer present. There is a need to obtain this information, as the persistence of such a relaxing and/or stimulating effect has a significant impact on the consumer experience, such as for example on whether the relaxing or stimulating effect of a scented shower gel is maintained after the show (i.e. after the event of applying the shower on a medium event-free term) and consequently on the creation and formulation of corresponding perfumed compositions and perfumed consumer products. So far, there is no reliable method for measuring such physiological effects on a medium (event-free) term.

Moreover, publications have disclosed that physiological responses (e.g., mean heart rate, electromyography, electrodermal activity) are different for an odor that is pleasant compared to one that is unpleasant. However, it has not been demonstrated that odors judged as pleasant and intense in a similar way can be distinguished on the basis of their physiological responses. There is a need to obtain this information, as odors judged as pleasant and intense in a similar way can have different physiological stimulating or relaxing effects which in turn has an impact on the creation and formulation of corresponding perfumed compositions and perfumed consumer products

Therefor, there is a need to reliably identify the relaxing and stimulating (energizing, invigorating) properties of materials having an odor in an event independent manner, i.e. not immediately after smelling but at medium term, and in particular for providing a reliable basis for designing relaxing and stimulating (energizing, invigorating) perfumes, perfuming compositions and perfumed consumer products.

The present invention provides a reliable method of identifying the relaxing and stimulating (energizing, invigorating) properties of materials having an odor in an event independent manner on a medium term.

### BRIEF DESCRIPTION OF THE FIGURES

FIG. 1 is a diagram showing the sequence of events for the presentation of one odor.

FIG. 2 shows graphs of: (A) raw PPG signal (blue circle=peak, green circle=minimum); (B) inter beat interval (ms); and (C) time series of PPG amplitudes (arbitrary unit).

FIG. 3 is a graph showing the correlation between ratings of relaxing/energizing properties of odors (abscissa) and ANS index values.

FIG. 4 is a graph showing mean (±SD) distribution density and individual data points of ANS index values for lavender and peppermint.

FIG. 5 is a graph showing mean (±SD) distribution density and individual data points of ANS index values for peppermint and no odor condition.

### DETAILED DESCRIPTION OF THE INVENTION

The present invention is directed on medium term effects using an index that reveals the relaxing/energizing (stimulating, invigorating) properties of odors similar in pleasantness and intensity rather than focusing on short term effects (event-related) during the seconds following olfactory stimulation. Thus, the present invention provides a reliable method for measuring physiological invigoration or relaxation effects of odors and aromas after a subject smells a fragrance on a medium term.

The expression medium term is herein preferably understood as a time period of at least 25 seconds. In a particular embodiment, the expression medium term is herein understood as a time period of at least 40 seconds and preferably at least 90 seconds. In a particular embodiment, the expression medium term is herein understood as the time period of not more than 600 seconds, preferably not more than 300 seconds, more preferably not more than 150 seconds, even more preferably not more than 90 seconds. In a particular embodiment, the expression medium term is herein understood as the time period of from 25 seconds to 600 seconds, preferably from 40 seconds to 300 seconds, even more preferably from 90 seconds to 150 seconds. In a particular embodiment, the expression is herein understood as a time period of 90 seconds.

The present invention is applicable to perfumery and aroma compositions, including blends, ingredients and essential oils. It applies to emotional benefits of a perfume or aroma, and enables the measurement of associated physiological changes.

The present invention relates to a method for identifying a material having an odor that increases the relaxed or stimulated physiological state in a subject, the method comprises the following steps:
a. letting the subject smell a device comprising a material having an odor,
b. measuring at least one body parameter indicative for the autonomic nervous system in the subject during its resting state;
c. relating the at least one body parameter to an index for a first state of the autonomic nervous system (first ANSind) of the subject;
d. conducting steps to a. to c. at least two additional times,
   wherein the subject is letting smelled at least one device comprising a material having no odor,
   wherein the at least one measured body parameter thereof is related to an index for a second state of the autonomic nervous system (second ANSind) of the subject;
e. identifying the material having an odor as relaxing or stimulating when the first ANSind of the material having an odor is indicative for being relaxing or stimulating over the second ANSind of the second material.

According to the present invention, in step a., a subject is letting smelled at a device comprising a material having an odor.

Thereby it is understood that a subject is letting smelled at a device comprising a (first) material having an odor.

A subject is herein preferably understood as a human being. In a particular embodiment, the subject is a healthy human. In a particular embodiment, the subject is a healthy human and free of psychiatric or neurological history. In a particular embodiment, the subject has a normal sense of smelling. In a particular embodiment, the subject is a healthy human and free of psychiatric or neurological history and has a normal sense of smell. In a particular embodiment, the subject did not eat and/or drink 4 hours, preferably 3 hours and more preferably not more than 2 hours before conducting the method.

In a particular embodiment, the subject is a group of subjects, preferably comprising at least 5, preferably at least 10 subjects, more preferably at least 15 subjects, and even more preferably at least 18 subjects, at least 20 subjects. The group of subjects preferably comprises a number of women and men in a ratio of 5:1 to 1:5, preferably 4:1 to 1:4, more preferably 3:1 to 1:3, more preferably 2:1 to 1:2.

A device is herein understood as means for providing a material so that a subject is able to smell the material. A device can be for example a container, a glass or plastic tube, a pen, a stripe, a probe, an olfactometer etc.

In a particular an embodiment, step a. is performed with an olfactometer. Any olfactometer can be used as long as it is compatible with measuring the parameters in step a. to enable the supply of defined, reproducible olfactory stimuli in the nose, without tactile or thermal stimulation, in a precise and controlled manner.

In a particular embodiment, the subject is letting smelled at the device for a segment of at least 1 second, preferably at least 5 seconds, more preferably at least 10 seconds and even more preferably at least 15 seconds. In a particular embodiment, the subject is letting smelled at the device for a segment of not more than 45 seconds, preferably not more than 35 seconds, more preferably not more than 25 seconds and even more preferably not more than 15 seconds. In a particular embodiment, the subject is letting smelled at the device for a segment of from 1 to 45 seconds, preferably from 5 to 35 seconds, more preferably from 10 to 25 seconds, even more preferably from 12 to 20 seconds and even more preferably for 15 seconds.

In a particular embodiment, the subject is letting smelled at the device several times each independently for a segment as described herein-above. In a particular embodiment, the subject is letting smelled at the device at least once, preferably at least twice and more preferably 3 times. In a particular embodiment, the subject is letting smelled at the device not more than 5 times, preferably not more than 4 times and more preferably not more than 3 times. In a particular embodiment, the subject is letting smelled at the device from 1 to 5 times, preferably 2 to 4 times and more preferably 3 times.

In a particular embodiment, between each segment of letting the subject smell the device, the odor is evaluated for intensity, liking, familiarity and/or relaxing/energizing properties. In a particular embodiment, the evaluation for intensity, liking, familiarity and/or relaxing/energizing properties is following the protocol as described for example in Porcherot et al., Food Quality and Preference, 2010; 21(8):938-947.

In a particular embodiment, the subject is letting smelled at the device for a total of at least 10 seconds, preferably at least 20 seconds, more preferably at least 30 seconds, even more preferably at least 40 seconds. In a particular embodiment, the subject is letting smelled at the device for not more than 90 seconds, preferably not more than 80 seconds, more preferably not more than 60 seconds, even more preferably not more than 50 seconds. In a particular embodiment, the subject is letting smelled at the device from 10 to 90 seconds, preferably from 20 to 80 seconds, more preferably from 30 to 60 seconds, even more preferably from 40 to 50 seconds, even more preferably for 45 seconds.

The material having an odor is herein preferably understood as a perfuming ingredient, perfume, perfuming composition or perfumed consumer product, preferably a perfuming ingredient.

By “perfume” (or also “perfume oil”) what is meant here is an ingredient or composition that is a liquid, solid or semi-solid at about 20° C. According to any one of the above embodiments said perfume oil can be a perfuming ingredient alone or a mixture of ingredients in the form of a perfuming composition. As a “perfuming ingredient” it is meant here a compound, which is used for the primary purpose of conferring or modulating an odor. In other words such an ingredient, to be considered as being a perfuming one, must be recognized by a person skilled in the art as being able to at least impart, enhance or modify the odor of a composition. For the purpose of the present invention, perfume oil also includes combination of perfuming ingredients with substances which together improve, enhance or modify the delivery of the perfuming ingredients, such as perfume precursors, emulsions or dispersions, as well as combinations which impart an additional benefit beyond that of modifying or imparting an odor, such as long-lasting, blooming, malodor counteraction, antimicrobial effect, microbial stability, insect control.

The nature and type of the perfuming ingredients do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said ingredients may also be compounds known to release in a controlled manner various types of perfuming compounds.

The perfuming ingredients may be dissolved in a solvent of current use in the perfume industry. Examples of such solvents are dipropylene glycol (DIPG), diethyl phthalate, isopropyl myristate, Abalyn® (rosin resins, available from Eastman), benzyl benzoate, ethyl citrate, limonene or other terpenes, or isoparaffins. Preferably, the solvent is very hydrophobic and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

According to the present invention, in step b., at least one body parameter indicative for the autonomic nervous system in the subject is measured during its resting state.

Thereby it is understood that at least one body parameter is measured which relates to the unconscious autonomic nervous system of a subject during the resting or task-negative state, i.e when an explicit task is not performed.

In a particular embodiment, the at least one body parameter is at least one parameter indicative for the sympathetic and/or parasympathetic nervous system.

In a particular embodiment, the at least one body parameter indicative for the autonomic nervous system in a subject can comprise:
i. at least one body parameter of the sympathetic nervous system (BP (SNS)) and/or
ii. at least one body parameter of the parasympathetic nervous system (BP(PNS))
   wherein BP(SNS) is a body parameter of the sympathetic nervous system and BP(PNS) is a body parameter of the parasympathetic nervous system.

In a particular embodiment, the at least one body parameter indicative for the autonomic nervous system comprises at least one parameter indicative for the sympathetic nervous system and at least one parameter of the parasympathetic nervous system. In a particular embodiment, the at least one body parameter comprises at least one parameter indicative for the sympathetic nervous system and at least two parameter of the parasympathetic nervous system. In a particular embodiment, the at least one body parameter comprises at least two parameter indicative for the sympathetic nervous system and at least one parameter of the parasympathetic nervous system. In a particular embodiment, the at least one body parameter comprises at least two parameter indicative for the sympathetic nervous system and at least two parameter of the parasympathetic nervous system. In a particular embodiment, the at least one body parameter comprises at least three parameter indicative for the sympathetic nervous system and at least two parameter of the parasympathetic nervous system. In a particular embodiment, the at least one body parameter comprises at least two parameter indicative for the sympathetic nervous system and at least three parameter of the parasympathetic nervous system.

In a particular embodiment, the at least one body parameter comprises at least 1 body parameters indicative for the autonomic nervous system of a subject, at least 2 body parameters indicative for the autonomic nervous system of a subject, at least 3 body parameters indicative for the autonomic nervous system of a subject, at least 4 body parameters indicative for the autonomic nervous system of a subject and at least 5 body parameters indicative for the autonomic nervous system of a subject. In a particular embodiment, the at least one body parameter comprises not more than 9 body parameters indicative for the autonomic nervous system of a subject, at least 8 body parameters indicative for the autonomic nervous system of a subject, at least 7 body parameters indicative for the autonomic nervous system of a subject, at least 6 body parameters indicative for the autonomic nervous system of a subject and at least 5 body parameters indicative for the autonomic nervous system of a subject. In a particular embodiment, the at least one body parameter comprises 1 to 9, preferably the at least one body parameter comprises 2 to 8 body parameters indicative for the autonomic nervous system of a subject, preferably the at least one body parameter comprises 3 to 7 body parameters indicative for the autonomic nervous system of a subject, preferably the at least one body parameter comprises 4 to 6 body parameters indicative for the autonomic nervous system of a subject, more preferably the at least one body parameter comprises about 5 body parameters indicative for the autonomic nervous system.

In a particular embodiment, the at least one body parameter is selected from the list consisting of heart rate (HR), low frequencies fluctuations in cardiac variability (LF), non-specific skin conductance responses (nsSCR), photoplethysmography amplitude of the pulse (PPGa), root mean squared successive differences in inter beat intervals (RMSSD), blood pressure (BP), pulse rate (PPGr), tonic level of skin conductance (SCL), preejection period (PEP), pulse transit time (PTT), pupil diameter, laser contrast imaging (LSCI) or analysis (LASCA), infrared thermography, skin temperature or any combination thereof.

Heart rate (HR) is herein understood as the number of beats per minute in a subject. HR is influenced by both parasympathetic (via cholinergic muscarinic receptors) and sympathetic (via β-adrenergic receptors) nervous system. Heart rate can be measured through a photoplethysmograph (PPG). Every time periods between successive pulse waves (inter-pulse intervals in see) are inverted, multiplied by sixty to obtain instantaneous heart rates in beats per minute. These values are then averaged over the period of interest.

Amplitude of the pulse (PPGa) is herein understood as the total height of a photoplethysmographic pulse wave. PPGa reflects the vasodilation/vasoconstriction of the peripheral vasculature (via α-adrenergic receptors). PPGa can be measured using a photoplethysmograph. The amplitude of every pulse waves (PPGa) is calculated (local maximum−previous local minimum). The resulting PPGa are averaged over the period of interest.

Root mean squared successive differences in inter beat intervals (RMSSD) is herein understood as the root mean square of successive differences between normal heartbeats (RMSSD). RMSSD characterizes more particularly parasympathetic, respiratory-mediated influences (via cholinergic receptors) and is obtained by first calculating every successive time difference between inter-pulse intervals in ms during the period of interest. Then, each of the values is squared and the result is averaged before the square root of the total is obtained. RRSMD can be measured and calculated using a photoplethysmograph (PPG).

Low frequencies fluctuations in cardiac variability (LF) refers to low-frequency oscillations (with a characteristic frequency of 0.04-0.15 Hz) in PPG inter-pulses intervals time series. LF characterizes more particularly parasympathetic baroreflex-mediated variations (via α,β-adrenergic receptors). LF is calculated by applying a frequency decomposition on the inter-pulse intervals time series measured with a photoplethysmograph and extracting the coefficients for the 0.04-0.15 Hz frequency band over the period of interest.

Non-specific skin conductance responses (nsSCR) refers to electrodermal activity that occurs in the absence of an identifiable eliciting stimulus. The number of nsSCR is derived from electrodermal activity characterizes sympathetic variations (cholinergic muscarinic receptors, Boucsein, 2014). nsSCR can be measured by any electrodermal recording device using direct constant voltage method.

Tonic level of skin conductance (SCL). Electrodermal activity reflects both slow varying tonic sympathetic activity and fast varying phasic sympathetic activity. Tonic activity can be expressed in units of electrodermal level (SCL), while phasic activity is expressed in units of electrodermal responses (EDR).

The preejection period (PEP) is the time elapsed between the electrical depolarization of the left ventricle (QRS on the ECG) and the beginning of ventricular ejection and represents the period of left ventricular contraction with the cardiac valves closed. PEP is influenced by sympathetic activity by way of beta1 adrenoreceptors and shortens under stimulation. PEP can be derived noninvasively from impedance cardiography, which converts changes in thoracic impedance (as measured by electrodes on the chest and neck) to changes in volume over time and allows tracking of volumetric changes such as those occurring during the cardiac cycle.

Pulse transit time (PTT): Surrogate index of blood pressure changes, sympathetically controlled. Pulse transit time (PTT) is a measurement of the time it takes for an arterial pulse wave to reach the periphery. PTT can be calculated from the finger photoplethysmograph (PPG) of the oxygen saturation monitor and the R-wave of the electrocardiogram (ECG) during a polysomnogram.

Pupil diameter: Pupillary response is a physiological response that varies the size of the pupil, via the optic and oculomotor cranial nerve. Parasympathetic activations induce constriction. Sympathetic activations induce dilation.

Laser Speckle Contrast Imaging (LSCI) or Analysis (LASCA). Laser speckle contrast imaging (LSCI) is a novel non-invasive microvascular imaging modality. It measures sympathetically mediated vasoconstriction.

Skin temperature is a measure of sympathetically mediated peripheral vasoconstriction

In a particular embodiment, the at least one body parameter relates to the following parameters:
i. heart rate (HR),
ii. low frequencies fluctuations in cardiac variability (LF),
iii. non-specific skin conductance responses (nsSCR),
iv. photoplethysmography amplitude of the pulse (PPGa), and/or
v. root mean squared successive differences in inter beat intervals (RMSSD).

The present invention thereby preferably encompasses body parameters for an index that reflects sympathetic nervous system (SNS) and parasympathetic nervous system (PNS) activations and thereby is able to identify the relaxation/energy state of the body in response to an odor. As a subject moves from a relaxed to a stimulated state, the values for example of HR and nSCR increase while the values of PPGa, RMSSD and LF decrease.

The resting state is herein understood as the state of a subject occurring in a resting or task-negative state, i.e when an explicit task is not performed. The resting state is an operational definition referring to a constant condition without imposed stimuli or other behaviorally salient events. In a particular embodiment, the subject is seated or lying and the environment does not impose stimuli or other behaviorally salient events. In a particular embodiment, the subject is seated or lying in a dark room.

In a particular embodiment, the measurement during the resting state relates to a task-negative time until a subject reaches its resting state. On whether a subject reached its resting state can be determined for example by asking on whether the subject was not following a task or by EEG or functional magnetic resonance imaging (fMRI).

In a particular embodiment, the measurement during the resting state relates to a task negative time period of at least 25 seconds, preferably of at least 40 seconds and more preferably of at least 90 seconds. the measurement during the resting state relates to a task negative time period of not more than 600 seconds, preferably not more than 300 seconds, more preferably not more than 150 seconds and even more preferably not more than 90 seconds. In a particular embodiment, the measurement during the resting state relates to a task negative time period of of from 25 seconds to 600 seconds, preferably from 40 seconds to 300 seconds, even more preferably from 90 seconds to 150 seconds. In a particular embodiment, the measurement during the resting state relates to a task negative time period of 90 seconds.

According to the present invention, in step c., the at least one body parameter is related to an index for a first state of the autonomic nervous system (first ANSind) of the subject.

Thereby it is understood that the at least one body parameter is used as a reference for the state of the autonomic nervous system of the subject.

In a particular embodiment, the index for the autonomic nervous system can be based on a calculation and averaging of the body parameters and may preferably also comprise a standard deviation.

In a particular embodiment, wherein the measured body parameter(s) is/are related to an index for a first autonomic nervous system by calculating the autonomic nervous system index (ANSind) according to formula (I):

$${ANSind}_{so} = \frac{1}{N} ( n \cdot \frac{BP {( \dot{∧} )}_{o} - \overline{BP {( \dot{∧} )}_{s}}}{σ {BP ( \dot{∧} )}_{s}} - n \cdot \frac{BP {( \dot{∨} )}_{o} - \overline{BP {( \dot{∨} )}_{s}}}{σ BP {( \dot{∨} )}_{s}} ) \qquad ( I )$$

where o is an odor, s is a subject, X̅X̅ is the averaged XX, σ the standard deviation of XX values across all odors for a particular panelist, BP(\dot{∧}) is a body parameter whose numerical value increases with sympathetic activation and/or decreases with parasympathetic activation, BP(\dot{∨}) is a body parameter whose numerical value decreases with sympathetic activation and/or increases with parasympathetic activation, n each independently the number of differing body parameters of the sympathetic and parasympathetic nervous system and N the sum of the number of n.

In a particular embodiment, step c. comprises calculating the first autonomic nervous system index (ANSind) according to formula (II):

$${ANSind}_{so} = \frac{1}{5} ( \frac{{HR}_{o} - \overline{{HR}_{s}}}{σ {HR}_{s}} + \frac{{nsSCR}_{o} - \overline{{nsSCR}_{s}}}{σ {nsSCR}_{s}} - \frac{{PPGa}_{o} - \overline{{PPGa}_{s}}}{σ {PPGa}_{s}} - \frac{{RMSSD}_{o} - \overline{{RMSSD}_{s}}}{σ {RMSSD}_{s}} - \frac{{LF}_{o} - \overline{{LF}_{s}}}{σ {LF}_{s}} ) , \qquad ( II )$$

where o is an odor, s is a subject, X̅X̅ is the averaged XX, σ the standard deviation of XX values across all odors for a particular panelist, HR is heart rate, LF is low frequencies fluctuations in cardiac variability, nsSCR is non-specific skin conductance responses, PPGa is photoplethysmography amplitude of the pulse, RMSSD is root mean squared successive differences in inter beat intervals as described herein-above.

The calculation of ANSind allows an assessment of the relaxed and the stimulated physiological state of a subject as the more the PNS is activated and the physiological state corresponds to relaxation, the lower the ANSind is and the more activated the SNS and the more energetic the state, the higher the ANSind is.

According to the present invention, in step d., steps a. to c. are conducted at least two additional times.

Thereby it is understood that in step d. the same subject and the same set of body parameters and the same set up for measuring the body parameters as mentioned step a., b. and c. are conducted at least two additional times. In a particular embodiment, the set of steps a. to c. are conducted at least two additional times.

The same definitions and embodiments as described herein above for step a., b. and c. apply mutatis mutandis for step d.

Steps a., b. and c. are used at least two additional times in order to obtain at least two data sets to allow normalization of the data to provide a reliable assessment on whether the material having an odor is stimulating or relaxing.

In a particular embodiment, the steps a. to c. are conducted at least 3 additional times, 5 additional times, 7 additional times or 9 additional times. The more often the steps a. to c. are conducted the more reliable is the assessment on whether a material is relaxing or stimulating.

In case the steps a. to c. are conducted at least 3 additional times, the subject is letting smelled at least one device comprising a material having no odor and at least two materials having an odor. Alternatively, in case the steps a. to c. are conducted at least 3 additional times, the subject is letting smelled at least two devices comprising a material having no odor and at least one materials having an odor.

In case the steps a. to c. are conducted at least 5 additional times, the subject is letting smelled at least one device comprising a material having no odor and at least 4 materials having an odor. Alternatively, in case the steps a. to c. are conducted at least three additional times, the subject is letting smelled at least two devices comprising a material having no odor and at least 3 materials having an odor.

In case the steps a. to c. are conducted at least 7 additional times, the subject is letting smelled at least one device comprising a material having no odor and at least 6 materials having an odor. Alternatively, in case the steps a. to c. are conducted at least three additional times, the subject is letting smelled at least two devices comprising a material having no odor and at least 5 materials having an odor.

In case the steps a. to c. are conducted at least 9 additional times, the subject is letting smelled at least one device comprising a material having no odor and at least 8 materials having an odor. Alternatively, in case the steps a. to c. are conducted at least three additional times, the subject is letting smelled at least two devices comprising a material having no odor and at least 7 materials having an odor.

The definitions and embodiments for the material having no odor and having an odor are the same as herein-below.

According to the invention, in step d. the subject is letting smelled at least one device comprising a material having no odor. Thereby, it is understood that in step d. when conducting steps a. to c. at least two additional times, one of steps a. is letting a subject smell at least one device comprising a material having no odor

Thereby it is herein understood that a subject is letting smelled at least one device (probe) with a material having no odor and another device (probe) with a (second) material having an odor or comprising a material having no odor. Thereby, at least two data sets are obtained by the present invention, i.e. a data set for a (first) material having an odor, at least one data set for a material having no odor and, optionally, at least one further data set of a (second) material having an odor. This in turn allow normalization of the data to provide a reliable assessment on whether the material having an odor is stimulating or relaxing.

The subject is letting smelled a device with a material having no odor and in particular evaluated its physiological effect to determine on whether the material having an odor as measured in steps a. to c. is more or less stimulating or relaxing. This is mainly because the probe with no odor functions as a reference of the resting state of the subject which has not been imposed with a task in form of an odor. In a particular embodiment, the material having no odor is air, preferably non-odorous air.

The subject is letting smelled a further device with a material having an odor or having no odor and in particular evaluated its physiological effect. By this further testing, additional data points for either a material having an odor or a material having no odor are generated which in turn allows normalization of the data to make a reliable assessment on whether the material having an odor is stimulating or relaxing.

In a particular embodiment, the (second) material having an odor is the same as the (first) material having an odor, i.e. the material having an odor as used in steps a. to c. In a particular embodiment, the (second) material having an odor is not the same as the (first) material having an odor, i.e. the material having an odor as used in steps a. to c. In case the (second) material having an odor is not the same as the (first) material having an odor, the (first) and (second) materials are similar in intensity, liking, familiarity and/or their relaxing/energizing properties as determined for example in Porcherot et al., Food Quality and Preference, 2010; 21(8):938-947. In a particular embodiment, the (second) material has no odor.

For the sake of clarity, the order of step d. in the method of the present invention in which steps a. to c. for the material having no odor is conducted is exchangeable with steps a. to c. for the (first) material having an odor. Thereby, it is understood that the steps a. to c. according to step d. for a material having no odor can be conducted prior to or after the steps a. to c. for the (first) material having an odor.

In a particular embodiment, steps a. to c. for a material having no odor are conducted before the steps a. to c. for the (first) material having an odor. In a particular embodiment, steps a. to c. for the material having no odor are conducted after the steps a. to c. for the (first) material having an odor.

In a particular embodiment, steps a. to c. for a material having no odor are conducted before the steps a. to c. for the (second) material having an odor or having no odor. In a particular embodiment, steps a. to c. for the material having no odor are conducted after the steps a. to c. for the (second) material having an odor or having no odor.

According to the invention, in step d. the at least one measured body parameter thereof is related to an index for a second state of the autonomic nervous system (second ANSind) of the subject.

Thereby it is understood that the at least one measured body parameter of either the material having no odor or the measured body parameter of the (second) material having an odor or having no odor is related to a second state of the autonomic nervous system (second ANSind) of the subject using the same measures as for first ANSind.

Thereby it is understood that a second state of the autonomic nervous system (second ANSind) of the subject is brought in relationship with the at least one body parameter obtained by the measurement for the material having no odor or the measured body parameter of the (second) material having an odor or no odor.

In a particular embodiment, a second ANSind is calculated according to formula (I) or (II).

Thereby it is understood that the same formula (I) or (II) according to step b. is used with the parameters obtained by the measurement for the material having no odor or the measured body parameter of the (second) material having an odor or no odor.

The same definitions and embodiments as described herein above for the (first) material having an odor apply mutatis mutandis for relating at least one measure body parameter for the material having no odor or the measured body parameter of the (second) material having an odor or not having an odor.

According to the invention, in step e., the material is identified as having an odor as relaxing or stimulating when the first ANSind of the material having an odor is indicative for being relaxing or stimulating over the second ANSind of the material obtained in step d.

Thereby it is understood that when comparing the first ANSind with the second ANSind the comparison thereof is indicative on whether the (first) material having an odor is stimulating or relaxing relative to a (second) material having an odor or not having an odor or on whether a (first) material having an odor is stimulating or relaxing absolutely by comparing it to a material having no odor.

In a particular embodiment, the material having an odor is identified as relaxing when the first ANSind is lower than the second ANSind.

Thereby it is understood that the (calculated) values of the first ANSind and the second ANSind are compared and depending on whether the second ANSind is higher than the first ANSind the odor is classified as relaxing.

In a particular embodiment, the material having an odor is identified as stimulating when the first ANSind is higher than the second ANSind.

Thereby it is understood that the (calculated) values of the first ANSind and the second ANSind are compared and depending on whether the second ANSind is lower than the first ANSind the odor is classified as stimulating.

In a particular embodiment for determining the relaxing or stimulating properties of an odor according to the present invention include:
Conduct a test on a series of subjects;
Present a minimum of 5 pleasant test odors to be tested;
Deliver each odor once, in a random sequence, for a total of 45 seconds in 15 second segments;
Between each segment, have the odor evaluated by the subject for intensity, liking, familiarity and then their relaxing/energizing properties using a 10 cm linear scale, where 0=not relevant at all and 10=extremely relevant. Porcherot et al., Food Quality and Preference, 2010; 21(8):938-947;
Follow these presentations and evaluations with a 90-second resting state;
During the resting state, measure the autonomic nervous system with a photoplethysmograph and electrodermal activity in the non-dominant hand;
Extract from the physiological signals the HR, nsSCR, PPGa, RMSSD and LF during the 90 seconds resting state;
Calculate the ANSind according to formula (II); and
Assign the relaxing/energizing properties of the odors according to the ANSind according to formula (II);
or,
Conduct a test on a series of subjects;
Present an odor to be tested and a no odor condition;
Deliver each odor once, balanced across subjects, for a total of 45 seconds in 15 second segments;
Between each segment, have the odor evaluated for intensity, liking, familiarity and then their relaxing/energizing properties using a 10 cm linear scale, where 0=not relevant at all and 10=extremely relevant;
Follow these presentations and evaluations with a 90-second resting state;
During the resting state, measure the autonomic nervous system with a photoplethysmograph and electrodermal activity in the non-dominant hand;
Extract from the physiological signals the HR, nsSCR, PPGa, RMSSD and LF during the 90 seconds resting state;
Use one or several of the above measures to infer SNS/PNS; and
Assign the relaxing/energizing properties of the odor as compared to the no odor condition.

The present invention also relates to a method for creating a perfume, perfuming composition or perfumed consumer product comprising a relaxing or stimulating material or accord, the method comprises the following steps:
a. identifying a material having an odor that increases the relaxed or stimulated physiological state in a subject as described herein-above,
b. incorporating the material in a perfume, perfuming composition or perfumed consumer product.

The same definitions and embodiments as described herein above for identifying a material having an odor that increases the relaxed state in a subject applies mutatis mutandis for this aspect of the present invention.

The material being identified as increasing the relaxed state in a subject is included by standard means for a skilled person into a perfume, perfuming composition or perfumed consumer product.

The present invention also relates to a method for increasing the level of relaxing or stimulating material or accord in a perfume, perfuming composition or perfumed consumer product, the method comprises replacing a first material having an odor and having a first ANSind with a second material having an odor and having a second ANSind wherein the first ANSind and second ANSind are measured according to the method as described herein-above and wherein the second ANSind of the second material is indicative for being more relaxed or stimulated over the first ANSind of the first material.

Thereby it is understood that as the second material having an odor with an ANSind being greater than the first ANSind of the first material replaces the first material in a perfume, perfuming composition or perfuming consumer product the resulting perfume, perfuming composition or perfuming consumer product comprising the second material having has an increased stimulating odor compared to the perfume, perfuming composition or perfuming consumer product comprising the first material.

The same definitions and embodiments as described herein above for identifying a material having an odor that increases the stimulated state in a subject over another material having an odor applies mutatis mutandis for this aspect of the present invention.

The present invention also relates to a use of the autonomic nervous system index (ANSind) and/or method as described herein-above for identifying a material as being relaxing or stimulating.

In a particular embodiment, the present invention is used for identifying peppermint as stimulating. In a particular further embodiment, the present invention is used for identifying lavender as relaxing.

The present invention is also used for identifying certain ingredients as being either increasing the stimulated or relaxing physiological state of a subject.

Therefore, the present invention also relates to the use of peppermint for increasing the stimulated physiological state in a subject.

The present invention also relates to a perfume, perfuming composition or perfumed consumer product comprising a relaxing or stimulating material or accord identified by the method as described herein-above.

In a particular embodiment, the perfuming consumer product is in the form of a fine fragrance product, a laundry care product, a home care product, a body care product, a skin care product, an air care product, or a hygiene product.

### EXAMPLES

### Example 1

Eighteen subjects (11 female) between the ages of 18-45 (M=26.5, SD=6.59) were recruited for the study. The subjects were right-handed, self-reported a normal sense of smell, and were free of any psychiatric or neurological history. Subjects were instructed not to eat or drink two hours before the study.

### Procedure

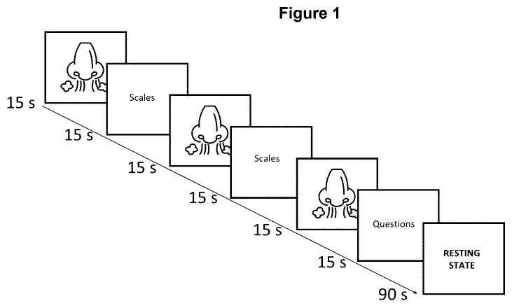

The order of the smells was pseudo-randomized for each subject. One smell was presented for a total of 45 seconds in each trial (FIG. 1), where every 15 seconds a 15 second break would occur and odorless air would be blown through the valves. During the break subjects were asked to answer questions about the properties of the smells. During the first two breaks, subjects were asked to answer four questions related to the smells using a linear scale how pleasant, familiar, intense, relaxing/energizing, the smell was. In the last 15 seconds, subjects were asked to rate how relaxed or energized they felt. All ratings were conducted on a sliding scale, from 0 to 100. Following this smell and question answering period, subjects were asked to let their thoughts wander for a 90 second resting state period. The screen would briefly flash at the end of the resting state to signal to subjects that the study would resume. For 50% of the trials, subjects were asked to rate what they had been thinking about during the resting state, with answers being: 1—thoughts provoked by the smell; 2—personal life; 3—the actual environment, like noise or the MRI; and 4—struggle against sleep. This procedure was repeated for each of the ten smells, in addition to one trial where air was used in the place of a smell, as a control condition. See FIG. 1.

### Stimuli

A total of 6 different odors were selected based on having the similar levels of pleasantness and intensity (how strong subjects thought the smell was). Air flow from the olfactometer was used as a neutral control condition. The odors used in the study, with their mean intensity, valence, and relaxing/energizing ratings, are presented in Table 1.

TABLE 1
Means' (SEM) assessments of valence (pleasantness/preference), intensity
and relaxing/energizing properties of the odors selected for the case study

| | ENERGIZING | FAMILIARITY | | INTENSITY | VALENCE |
|---|---|---|---|---|---|
| Lavender | 42.8 (5.2) | 70.0 | (6.7) | 70.9 (5.2) | 58.1 (6.6) |
| Magnolia | 45.5 (5.9) | 78.9 | (4.4) | 58.6 (5.5) | 49.9 (6.3) |
| Peach | 50.3 (5.9) | 64.5 | (7) | 52.8 (5.5) | 72.3 (4.7) |
| Strawberry | 49.3 (6.2) | 65.4 | (7.4) | 61.7 (3.6) | 68.9 (4.6) |
| Grapefruit | 61.8 (4.1) | 56.1 | (6.3) | 65.1 (4.2) | 60.3 (4.1) |
| Peppermint | 66.6 (5.7) | 67.3 | (7) | 60.9 (4.2) | 67.8 (5.5) |

### Stimuli Presentation

Odors were delivered by an MRI-compatible olfactometer. Ischer, et al., Frontiers in Psychology, 2014; 5(JUL). Odor-containing glass tubes were placed on a plastic support close to the participant. Odors were connected to a tube which was attached to an intranasal cannula. Each glass vial was pressure fed by a computer controlled air valve, which was switched on and off to send different odorant stimuli. During the neutral, no-odor, condition extra inter-stimulus air vales sent clear air to the nose. The system was connected to a separate air supply of the building and enabled a constant clean delivery of air, with no detectable flow variation when an odor was sent.

### Physiological Recordings and Preprocessing

Physiological activities were recorded (1000 Hz sampling frequency) using the Biopac Systems Inc. (BIOPAC Systems Inc, CA, USA) data acquisition system (MP150). The pulse (PPG) was measured using a plethysmograph attached on the distal phalanx of the ring finger of the left hand. The PPG signals were filtered offline with a Blackman −61 dB octave/slope filter (High-pass=0.5 Hz, Low-pass=4 Hz). Electrodermal activity (EDA) was measured with Beckman Ag—AgCl electrodes (8-mm diameter active area) filled with a skin conductance paste (Biopac) attached to subjects' non-dominant hand on the palmar side of the middle phalanges of the second and third fingers. The EDA signals were filtered offline with a Blackman −61 dB octave/slope filter (Low-pass=5 Hz).

### Measures Extraction

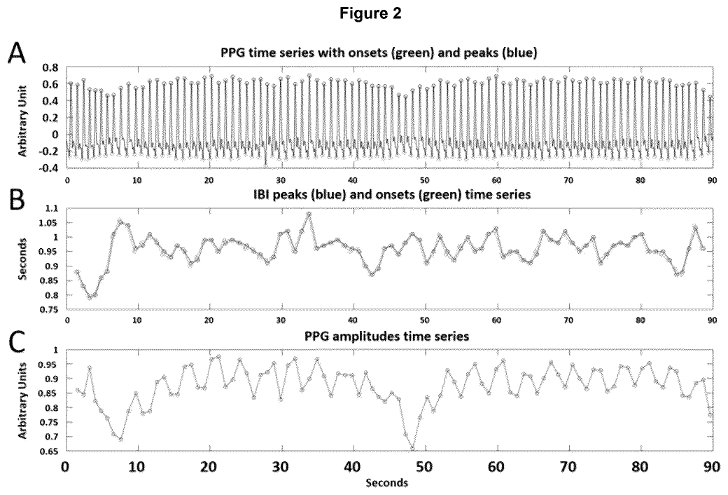

From the filtered PPG signal (FIG. 2A), the peaks and the interval (in seconds) between two consecutive peaks are calculated by standard software available for example from Biopac Systems Inc. or Kubios to form a time series of inter beat interval (IBI, FIG. 2B), as well as the amplitude that separated a peak from the previous minimum (PPGa, FIG. 2C). The IBI time series obtained for each 90-second periods of resting states was then analyzed with the HRVAS software to give the RMSSD, LF and HR values. The mean PPGa was obtained by averaging each PPGa values during the 90-second resting-state periods. See FIG. 2.

Each 90-second period of EDA signal was analyzed using Ledalab software to obtain the number of non-specific electrodermal responses (nsSCR) during the resting state.

The relationship between the relaxing/stimulating properties of odors reported by subjects and physiological measures of relaxation/energy was examined by correlating subjects' ratings measured using a 10 cm linear scale, where 0=not relevant at all and 10=extremely relevant and the ANS index.

The index performs particularly well if odors that are judged to be similar in pleasantness/preference and intensity show statistical differences in physiological ANS activations and subjects' ratings of relaxing/energizing properties. For this purpose, paired measures t-tests were performed to test statistical differences between the different variables.

### Results

### Correlation Between Relaxing/Energizing Ratings and ANS Index

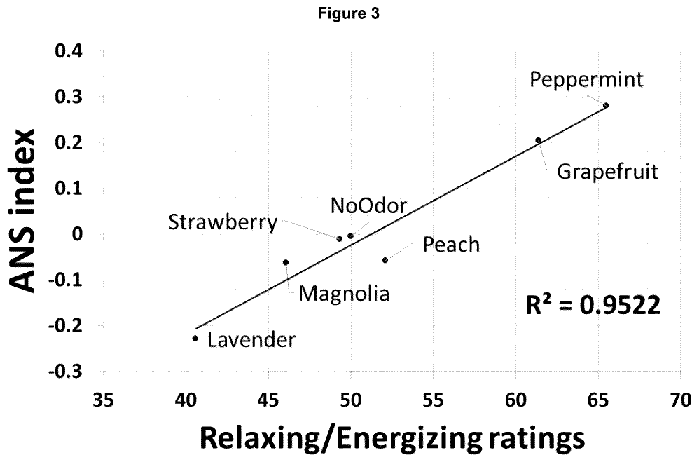

To assess the extent to which the physiological measure of relaxation/energy are related to judgments of the relaxing/energizing nature of odors, the correlation between the ANS index and subjects' relaxing/energizing ratings obtained from using a 10 cm linear scale, where 0=not relevant at all and 10=extremely relevant was calculated (FIG. 3).

The results (Table 2) show a strong correlation between the ANS index and evaluations of the relaxing/stimulating character of odors.

TABLE 2
Pearson and Spearman correlations between relaxing/energizing ratings and ANS index:

| | | | | Pearson | | | | Spearman | | | |
|---|---|---|---|---|---|---|---|---|---|---|---|
| | | | n | r | p | Lower 95% CI | Upper 95% CI | [symbol] | p | Lower 95% CI | Upper 95% CI |
| ENERGIZING | — | ANSind | 7 | 0.97 [symbol] | 1.724e−4 | 0.840 | 0. [symbol] 97 | 0.893 | 0.012 | 0.427 | 0. [symbol] 84 |

[symbol]

indicates data missing or illegible when filed

Potential influences of odor valence and intensity were then controlled for, and the partial correlation performed (Table 3) also showed a strong positive relationship between the two variables. This ruled out the possibility that the results were due to pleasantness and odor intensity.

TABLE 3
Partial Pearson and Spearman correlations between
relaxing/energizing ratings and ANS index:

| | | | | Pearson | | Spearman | |
|---|---|---|---|---|---|---|---|
| | | | n | r | p | [symbol] | p |
| ENERGIZING | — | ANSind | 7 | 0.987 | 0.002 | 0.924 | 0.025 |

Note [symbol]
Conditioned on variables: VALENCE, INTENSITY. [symbol]
indicates data missing or illegible when filed

### Relaxing/Energizing Properties of Odors Similar in Intensity and Valence

The following analyses showed that with this invention odors that are similar in intensity and preference and yet have different relaxing/energizing properties can be differentiated. Subject ratings and physiology for peppermint and lavender odors were used for comparison.

The peppermint and lavender odors were not differentiated by subjects on the basis of liking or intensity evaluations as shown in Tables 4 and 5. The t-tests were not significant and the Bayes factor (BF₀₁=1/BF₁₀) calculation revealed that the probability that there was no difference on these variables was at least times greater than the probability that there were differences.

TABLE 4
Two tailed paired t-test between lavender and peppermint liking ratings:

| | | | | | | | | | 95% Confidence Interval | |
|---|---|---|---|---|---|---|---|---|---|---|
| | | | Statistic | [symbol] % | df | p | | Effect Size | Lower | Upper |
| 01_Lavender | 10_Peppermint | Student's t | −1.2504 | | 17.000 | 0.228 | Cohen's d | −0.29471 | −0.76291 | 0.18172 |
| | | Bayes factor [symbol] | 0.47636 | 2.0526e−4 | | | | | | |

[symbol]

indicates data missing or illegible when filed

TABLE 5
Two tailed paired t-test between lavender and peppermint intensity ratings.

| | | | | | | | | | 95% Confidence Interval | |
|---|---|---|---|---|---|---|---|---|---|---|
| | | | Statistic | [symbol] % | df | p | | Effect Size | Lower | Upper |
| 01_Lavender | 10_Peppermint | Student's t | 1.5711 | | 17.000 | 0. [symbol] 35 | Cohen's d | 0.37030 | −0.11294 | 0. [symbol] |
| | | Bayes factor [symbol] | 0.68577 | 1.6551e−5 | | | | | | |

[symbol]

indicates data missing or illegible when filed

The t-test performed on the relaxant/stimulant ratings was statistically significant (Table 6), showing that the subjects rated peppermint as more energizing than lavender.

TABLE 6
Two tailed paired t-test between lavender and peppermint relaxing/energizing ratings:

| | | | | | | | | | 95% Confidence Interval | |
|---|---|---|---|---|---|---|---|---|---|---|
| | | | Statistic | [symbol] % | df | p | | Effect Size | Lower | Upper |
| 01_Lavender | 10_Peppermint | Student's t | − [symbol] | | 17.000 | 0.001 | Cohen's d | − [symbol] 0. | −1.4 [symbol] | −0.3 [symbol] |
| | | Bayes factor [symbol] | 27. [symbol] | [symbol] | | | | | | |

[symbol]

indicates data missing or illegible when filed

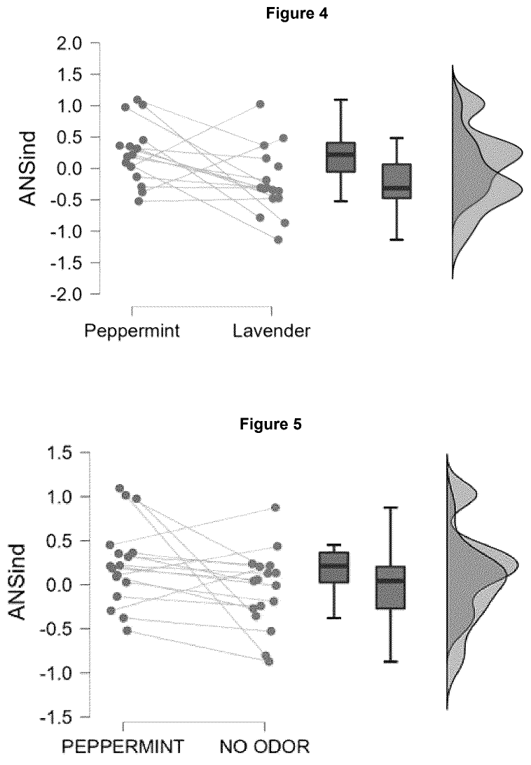

The ANS index was statistically lower during the resting state following the lavender presentation than following the peppermint presentation (Table 7 and FIG. 4).

TABLE 7
One tailed paired t-test between ANS index for lavender and peppermint:

| | | | | | | | | | 95% Confidence Interval | |
|---|---|---|---|---|---|---|---|---|---|---|
| | | | Statistic | [symbol] % | df | p | | Effect Size | Lower | Upper |
| 01_Lavender | 10_Peppermint | Student's t | [symbol] | | 14.000 | 0.020 | Cohen's d | [symbol] | [symbol] | [symbol] |
| | | Bayes factor [symbol] | [symbol] | [symbol] | | | | | | |

No [symbol] [symbol]
indicates data missing or illegible when filed

The results show that the emotional physiological benefit of a fragrance may be characterized in terms of relaxation/stimulating, in the medium term, for odors that are similar in pleasantness and intensity by the combination of a resting-state protocol, physiological measurements and the calculation of an ANS index according to the present invention.

Moreover, the ANS index was statistically lower during the resting state following no odor presentation than following the peppermint presentation (Table 8 and FIG. 5).

TABLE 8
One tailed paired t-test between ANS index
for no odor (NONE) and peppermint (PEPP):

| | | | | | | | 95% Confidence Interval | |
|---|---|---|---|---|---|---|---|---|
| Measure 1 | | Measure 2 | t | df | p | Cohen's d | Lower | Upper |
| NONE | — | PEPP | −2.0 [symbol] 7 | 16 | 0.029 | −0.49 [symbol] | [symbol] | [symbol] |

[symbol]

Note [symbol]
Student's t-test [symbol] [symbol]
indicates data missing or illegible when filed

The results show that the emotional physiological benefit of a fragrance may be characterized in absolute terms of relaxation/stimulating, in the medium term, by the combination of a resting-state protocol, physiological measurements and the calculation of an ANS index according to the present invention.

1. A method for identifying a material having an odor that increases the relaxed or stimulated physiological state in a subject, the method comprises the following steps:
a. letting the subject smell a device comprising a material having an odor,
b. measuring at least one body parameter indicative for the autonomic nervous system in the subject during its resting state;
c. relating the at least one body parameter to an index for a first state of the autonomic nervous system (first ANSind) of the subject;
d. conducting steps a. to c. at least two additional times,
wherein the subject is letting smelled at least one device comprising a material having no odor,
wherein the at least one measured body parameter thereof is related to an index for a second state of the autonomic nervous system (second ANSind) of the subject;
e. identifying the material having an odor as relaxing or stimulating when the first ANSind of the material having an odor is indicative for being relaxing or stimulating over the second ANSind of the material obtained in step d.

2. The method according to claim 1, wherein the at least one body parameter indicative for the autonomic nervous system in the subject comprises at least one parameter indicative for the sympathetic nervous system and at least one parameter of the parasympathetic nervous system.

3. The method according to claim 1, wherein at least one of the following body parameters indicative for the autonomic nervous system in a subject are measured:
i. at least one body parameter of the sympathetic nervous system and/or
ii. at least one body parameter of the parasympathetic nervous system
and
wherein the measured body parameter(s) is/are related to an index for a first and second state of the autonomic nervous system by calculating the autonomic nervous system index (ANSind) according to formula (I):
$${ANSind}_{so} = \frac{1}{N} ( n \cdot \frac{BP {( \dot{∧} )}_{o} - \overline{BP {( \dot{∧} )}_{s}}}{σ {BP ( \dot{∧} )}_{s}} - n \cdot \frac{BP {( \dot{∨} )}_{o} - \overline{BP {( \dot{∨} )}_{s}}}{σ BP {( \dot{∨} )}_{s}} ) \qquad ( I )$$
where o is an odor, s is a subject, X̅X̅ is the averaged XX, σ the standard deviation of XX values across all odors for a particular panelist, BP(\dot{∧}) is a body parameter whose numerical value increases with sympathetic activation (under sympathetic influences) and/or decreases with parasympathetic activation (under parasympathetic influences), BP(\dot{∨}) is a body parameter whose numerical value decreases with sympathetic activation (under sympathetic influences) and/or increases with parasympathetic activation (under parasympathetic influences), n each independently the number of differing body parameters of the sympathetic and parasympathetic nervous system and N the sum of the number of n.

4. The method according to claim 1, wherein in step b. at least three body parameters indicative for the autonomic nervous system are measured.

5. The method according to claim 1, wherein the following body parameters indicative for the autonomic nervous system in a subject are measured:
i. heart rate (HR),
ii. low frequencies fluctuations in cardiac variability (LF),
iii. non-specific skin conductance responses (nsSCR),
iv. photoplethysmography amplitude of the pulse (PPGa),
v. root mean squared successive differences in inter beat intervals (RMSSD); and
wherein the body parameters are related to an index for a first and second state of the autonomic nervous system by calculating the autonomic nervous system index (ANSind) according to formula (II):
$${ANSind}_{so} = \frac{1}{5} ( \frac{{HR}_{o} - \overline{{HR}_{s}}}{σ {HR}_{s}} + \frac{{nsSCR}_{o} - \overline{{nsSCR}_{s}}}{σ {nsSCR}_{s}} - \frac{{PPGa}_{o} - \overline{{PPGa}_{s}}}{σ {PPGa}_{s}} - \frac{{RMSSD}_{o} - \overline{{RMSSD}_{s}}}{σ {RMSSD}_{s}} - \frac{{LF}_{o} - \overline{{LF}_{s}}}{σ {LF}_{s}} )$$
where o is an odor, s is a subject, XX is the averaged XX and σ the standard deviation of XX values across all odors for a particular panelist.

6. The method according to claim 1, wherein the material is identified as relaxing when the first ANSind is lower than the second ANSind and wherein the material is identified as stimulating when the first ANSind is higher than the second ANSind.

7. The method according to claim 1, wherein the measurement during the resting state in step b. relates to a task-negative time until a subject reaches its resting state.

8. The method according to claim 1, wherein the measurement during the resting state in step b. relates to a task negative time period of from 25 seconds to 600 seconds, preferably from 40 seconds to 300 seconds, even more preferably from 90 seconds to 150 seconds, most preferably about 90 seconds.

9. A method for creating a perfume, perfuming composition or perfumed consumer product comprising a relaxing or stimulating material or accord, the method comprises the following steps:
a. identifying a material having an odor that increases the relaxed or stimulated physiological state in a subject according to claim 1,
b. incorporating the material in a perfume, perfuming composition or perfumed consumer product.

10. A method for increasing the level of relaxing or stimulating material or accord in a perfume, perfuming composition or perfumed consumer product, the method comprises replacing a first material having an odor and having a first ANSind with a second material having an odor and having a second ANSind wherein the first ANSind and second ANSind are measured according to the method according to claim 1 and wherein the second ANSind of the second material is indicative for being more relaxed or stimulated over the first ANSind of the first material.

11. The method according to claim 1, comprising identifying the material as being stimulating.

12. The method according to claim 11, wherein the material is peppermint.

13. A method of increasing the stimulated physiological state in a subject, comprising exposing the subject to a peppermint odor at a stimulating intensity.

14. A perfume, perfuming composition or perfumed consumer product comprising a relaxing or stimulating material or accord identified by the method of claim 1.

15. A perfume, perfuming composition or perfume consumer product according to claim 14, wherein the perfume, perfuming composition or perfumed consumer product is in the form of a fine fragrance product, a laundry care product, a home care product, a body care product, a skin care product, an air care product, or a hygiene product.
